(19) United States
(12) Patent Application Publication
(10) Pub. No.: US 2007/0239103 A1
(43) Pub. Date: Oct. 11, 2007

(54) Stylet markings

(76) Inventors: David M. Hardin, Winston-Salem, NC (US); Victor D. Clark JR., Winston-Salem, NC (US); Rig S. Patel, Raleigh, NC (US)

Correspondence Address: BRINKS HOFER GILSON & LIONE/CHICAGO/COOK, PO BOX 10395, CHICAGO, IL, 60610, US

(21) Appl. No.: 11/635,267

(22) Filed: Dec. 7, 2006

Related U.S. Application Data
(60) Provisional application No. 60/749,075, filed on Dec. 8, 2005.

(51) Int. Cl. A61M 1/00

(52) U.S. Cl. 604/27

(57) ABSTRACT

The present invention provides a medical system comprising an elongated medical device having a longitudinal lumen, such as a catheter or a needle, and a stylet configured to be inserted into and subsequently removed from the longitudinal lumen. The stylet is configured to have at least one indicator that indicates that the distal end of the stylet is about to exit the lumen as the stylet is being withdrawn during use. Each indicator alerts the user that the stylet is almost completely withdrawn from the lumen such that the user may take proper precautions and prevent the stylet from inadvertently and uncontrollably exiting the lumen. Each indicator may be located in the proximity of the distal end of the stylet and present either a visual, physical, or audible indication. Additionally, the present invention provides a method that indicates the longitudinal position of a stylet relative to an elongated medical device.

### PRIORITY AND CROSS-REFERENCE TO RELATED APPLICATION

This application claims priority to provisional application Ser. No. 60/749,075, filed on Dec. 8, 2005, which is incorporated by reference in its entirety herein.

### FIELD OF THE INVENTION

The present invention relates generally to the field of medical stylets. More particularly, the invention relates to a stylet for use within a medical device, such as a needle or a catheter.

### BACKGROUND OF THE INVENTION

Stylets have been used with medical devices, such as needles and catheters, to control the proper maneuvering of the medical device during and after the introduction of that device into a patient's body. The medical devices typically have an interior longitudinal lumen into which a stylet may be inserted. The stylet may have a handling mechanism at one end that medical personnel use to longitudinally move the stylet within the lumen of the medical device.

During the use of a conventional stylet, medical personnel withdraw the stylet from the medical device after the medical device is introduced into the body of a patient. As the stylet is withdrawn from the medical device, the distal end of the stylet approaches the proximal end of the lumen. Eventually, the stylet is entirely withdrawn from the lumen. However, as the distal end of the stylet exits the lumen, a number of disadvantages may arise.

Conventional stylets and medical devices do not have any indicator that indicates, as the stylet is being withdrawn from the lumen, that the distal end of the stylet is about to exit the lumen. In other words, the medical personnel withdrawing the stylet from the lumen have no mechanism that alerts them that the stylet is about to literally “pop out of” the lumen. Hence, the medical personnel removing the stylet from the lumen may be caught off guard when the distal end of the stylet exits the lumen.

For example, when the stylet suddenly pops out of the lumen, the unprepared medical personnel may not have the distal end of the stylet physically under control. As a result, the unwieldy distal end of the stylet may uncontrollably swing around and inadvertently make contact with either medical personnel or the patient. The unwieldy distal end of the stylet also may spread and disperse droplets of bodily fluid as it randomly swings around. Additionally, when the distal end of the stylet exits the lumen, bodily fluids may unwantedly drain from the lumen in an unpreventable manner.

The present invention alleviates one or more of the shortcomings described above.

### BRIEF SUMMARY

The present invention provides a stylet for use within a medical device, such as a catheter or a needle. The medical device may have an interior longitudinal lumen. The exterior of the stylet may be sized to be insertable and longitudinally movable within the lumen such that the stylet may be used to control and maneuver the medical device as desired during and after the introduction of the medical device into the body of a patient. During use, the stylet may be completely withdrawn or removed from the lumen. At least one indicator or marker is provided on the stylet to alert medical personnel that the stylet is almost completely removed from the lumen as the stylet is being withdrawn from the lumen.

In one embodiment of the present invention, a medical system may include an elongated medical device having a longitudinal lumen extending through a portion thereof and a stylet sized to be insertable and longitudinally movable within the lumen. The stylet is configured to have at least one indicator located approximately in the proximity of its distal end that indicates that the stylet is about to be completely withdrawn from the lumen as the stylet is being removed from the lumen during use. The types of indicators may include ink markings, laser etchings, changes in the material type of the stylet, holes, indentations into the exterior of the stylet, sandblastings of at least a portion of the exterior of the stylet, coatings of at least a portion of the exterior of the stylet, or reductions or expansions in the outer diameter of the stylet.

In another embodiment of the present invention, a medical device may include a stylet having a proximal end and a distal end, the stylet being configured to be insertable and longitudinally movable within a longitudinal lumen of an elongated medical apparatus. The stylet has at least one indicator located in the approximate proximity of the distal end of the stylet that indicates that the stylet is about to be completely withdrawn from the lumen as the stylet is being removed from the lumen during use. The indicator may provide at least one visual, physical, or audible indication.

In another embodiment of the present invention, a method for indicating the longitudinal position of a stylet relative to an elongated medical device may include at least partially withdrawing a stylet having a proximal end and a distal end from a longitudinal lumen of an elongated medical device and recognizing at least one indication provided by an indicator located on the stylet in the approximate proximity of the distal end. The method may include recognizing visual, physical, or audible indications.

Advantages of the present invention will become more apparent to those skilled in the art from the following description of the preferred embodiments of the invention which have been shown and described by way of illustration. As will be realized, the invention is capable of other and different embodiments, and its details are capable of modification in various respects. Accordingly, the drawings and description are to be regarded as illustrative in nature and not as restrictive.

### BRIEF DESCRIPTION OF THE DRAWINGS

FIG. 1 is a longitudinal cross-sectional view of one embodiment of the medical device of the present invention;

FIG. 2 is a cross-sectional view of a portion of one embodiment of the medical device of the present invention;

FIG. 3 is a schematic illustration of one type of indicator of the present invention;

FIG. 4 is a schematic illustration of another type of indicator of the present invention;

FIG. 5 is a schematic illustration of two types of indicators of the present invention; and

FIG. 6 is a schematic illustration of another type of indicator of the present invention.

### DETAILED DESCRIPTION

In accordance with the present invention, a stylet for use within a medical device, such as a needle, a catheter, or other cannula-type device, is provided. The medical device may have an interior longitudinal lumen. The stylet may be sized to be insertable and longitudinally movable within the lumen of the medical device such that the stylet may be used to control and maneuver the medical device as desired during the introduction of the medical device into the body of a patient during a medical procedure. The stylet may be movable via a mechanism attached to one end, such as a handle or a knob.

During the medical procedure, the stylet may be completely withdrawn or removed from the lumen. The stylet is configured to have an indicator that indicates to a user that, as the user is withdrawing the stylet from the lumen during use, the stylet is about to be completely withdrawn from the lumen. The indicator alerts the user that proper precautions should be taken regarding the further operation and handling of the medical device and stylet. The indicator may provide one or more visual, physical, or audible indications.

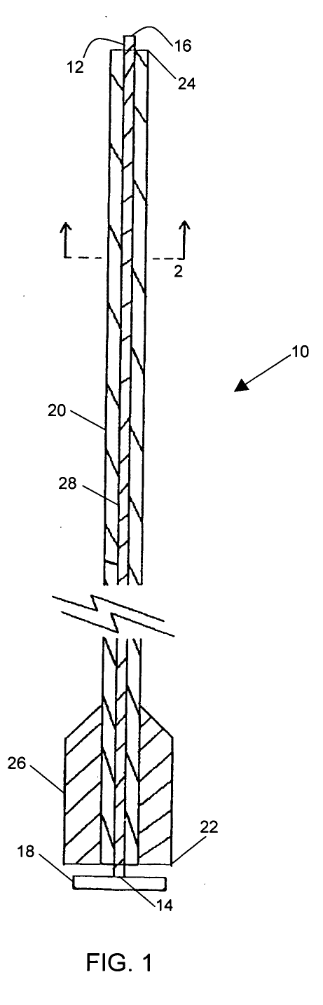

FIG. 1 illustrates one embodiment of the medical device 10 provided by the present invention. A stylet 12 may be sized to be insertable and longitudinally movable within the medical device 10. As shown, the stylet 12 is cylindrically shaped and has a long and slender, wire-like body. The stylet 12 has a proximal end 14 and a distal end 16. The stylet 12 also may have a knob 18 or other handling mechanism attached to the proximal end 14. In one aspect, the stylet 12 may be manufactured from metal, such as stainless steel, plastic, a polymer, or a copolymer.

The medical device 10 has a primarily tubular body 20. The tubular body 20 has a cylindrical exterior and is long and slender, similar in shape to the body of a conventional needle or catheter. As such, the tubular body 20 has a proximal end 22 and a distal end 24. In one aspect, the tubular body 20 may be manufactured from metal, plastic, a polymer, a copolymer, or some other flexible material.

The medical device 10 may have a handle 26 or other handling mechanism that facilitates enhanced control over the medical device 10 during use. The medical device 10 also has an interior longitudinal lumen 28. The lumen 28 is sized such that the stylet 12 may be inserted into, and subsequently movable along the longitudinal axis of, the lumen 28. The handle 26 also may have a cavity sized such that the stylet 12 may be inserted through the interior of the handle 26 and subsequently movable along the longitudinal axis of the handle 26.

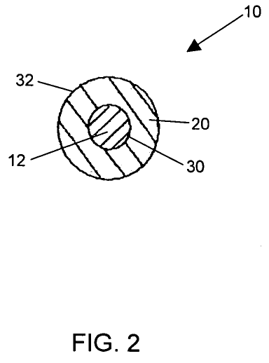

FIG. 2 illustrates a cross-sectional view of a portion of one embodiment of the medical device 10 of the present invention. The interior surface 30 of the tubular body 20 is generally cylindrical shaped and forms the boundary of the lumen 28. The stylet 12 has a generally cylindrical exterior with a circular cross-section. The stylet 12 may make either an interference or clearance fit with the interior surface 30 of the tubular body 20.

The exterior surface 32 of the tubular body 20 also has a generally cylindrical shape. The tubular body 20 may have an annular cross-section, as defined by the exterior surface 32 and the interior surface 30.

During the introduction of the medical device 10 into the body of a patient, the distal end 16 of the stylet 12 may protrude from the distal end 24 of the tubular body 20. The stylet 12 helps steer the distal end 24 of the tubular body 20 to the desired location. After introduction, a user may grip the knob 18 with one hand and the handle 26 with the other. The user may withdraw the stylet 12 from the lumen 28 by pulling on the knob 18. As the stylet 12 is withdrawn, the distal end 16 of the stylet 12 approaches the proximal end 22 of the tubular body 20. The medical device 10 may have other configurations including those with fewer or additional components.

The stylet 12 has at least one indicator or marker in the vicinity of the distal end 16. Consequently, as a user withdraws the stylet 12 from the lumen 28, the indicator or marker will exit the proximal end 22 of the tubular body 20 or the lumen 28 before the distal end 16 of the stylet 12. As a result, the user may be alerted by the indicator before the distal end 16 of the stylet 12 exits the lumen 28. Accordingly, the user may be permitted to take the appropriate precautions to prevent the premature complete withdrawal of the stylet 12 from the lumen 28, which may result in the unwieldy distal end of the stylet 12 swinging uncontrollably, possibly inadvertently making contact with either medical personnel or patients, or unwantedly spreading droplets of bodily fluid, as well as the premature drainage of bodily fluids from the lumen 28.

Therefore, in one aspect of the present invention, the stylet has an indicator that provides an indication that the stylet is about to be completely withdrawn from the lumen during use. The indicator may be located in the proximity of the distal end of the stylet. The indicator may include (1) an ink marking, (2) a laser etching, (3) a change in the material type of the stylet, (4) a hole, (5) an indentation into the exterior of the stylet, (6) sandblasting at least a portion of the exterior of the stylet, (7) coating at least a portion of the exterior of the stylet, or (8) a reduction or expansion in the outer diameter of the stylet.

In another aspect of the present invention, the user may be visually alerted that the distal end of the stylet is in close proximity to proximal end of the lumen. The stylet may have a visual indicator located in the proximity of the distal end of the stylet. In operation, as the user removes the stylet from the lumen, the visual indicator may provide a visual indication to the user that the stylet is approaching the point at which the stylet will be completely withdrawn from the lumen.

In another aspect of the present invention, the user may be physically alerted that the distal end of the stylet is in close proximity to proximal end of the lumen. The stylet may have a physical indicator located in the proximity of the distal end of the stylet. In operation, as the user removes the stylet from the lumen, the physical indicator may provide a physical indication to the user that the stylet is approaching the point at which the stylet will be completely withdrawn from the lumen.

In another aspect of the present invention, the user may be audibly alerted that the distal end of the stylet is in close proximity to the proximal end of the lumen. The stylet may have an audible indicator located in the proximity of the distal end of the stylet. In operation, as the user removes the stylet from the lumen, the audible indicator may provide an audible indication to the user that the stylet is approaching the point at which the stylet will be completely withdrawn from the lumen.

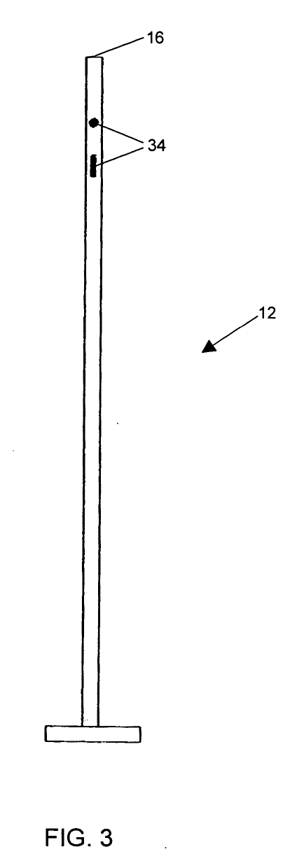

FIG. 3 illustrates one type of indicator of the present invention. As shown, the indicator may be an ink marking 34. The ink marking 34 may be either temporary, indelible, or permanent. For example, the stylet 12 may be marked with either waterproof or India ink. The ink may be of different colors, such as blue, black, red, yellow, etc. However, the color should be in contrast to the color of the remaining exterior of the stylet 12.

Additionally, the ink marking 34 may be of a distinct shape, such as a square, a rectangle, a circle, a pentagon, etc. The various shape of each ink marking 34 also may have a different size. As a result of the foregoing, an ink marking 34 may be visually recognized, i.e., seen, by a user. Accordingly, an ink marking 34 may provide a visual indication to the user that the distal end 16 of the stylet 12 is in close proximity to the proximal end of the tubular body.

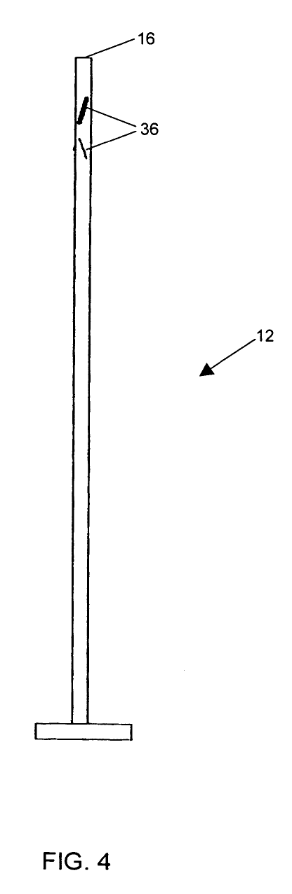

FIG. 4 illustrates another type of indicator of the present invention. As shown, the indicator may be a laser etching 36. The process of laser etching, also known as laser engraving, marking, or scribing, may entail using a laser etcher to develop a laser etching 36 on the exterior of the stylet 12. The laser etcher may be capable of etching different font characters, such as letters and numbers, shapes, lines, and logos. The laser etcher also may provide for adjustable depth and darkness of the resulting laser etching 36. As such, a laser etching 36 may be visually recognized by a user. Accordingly, a laser etching 36 may provide a visual indication to the user that the distal end 16 of the stylet 12 is in close proximity to the proximal end of the lumen.

Another type of indicator of the present invention may be a change in the material type of the stylet. The stylet may be made from various types of materials, such as metal or plastic. A change in the material type of the exterior of the stylet may alter the roughness or abrasiveness of the exterior or vary the stiffness of the stylet. A change in the material type also may vary the color of the stylet exterior. Hence, a different type of stylet material may provide a noticeable change in the exterior of the stylet that may be visually or physically recognized, i.e., seen or felt, by a user.

Moreover, the change in the exterior of the stylet may create a noticeable difference in the sound that is produced from the withdrawal of the stylet from the tubular body. In other words, the removal of the stylet from the lumen may result in noise audibly recognizable, i.e., heard, by a user. For example, if there is an interference fit between the stylet exterior and the interior of the tubular body, a change from smooth to extremely abrasive abrasive material on the stylet exterior, or vice versa, may increase or decrease the level or change the tone of the sound produced from withdrawing the stylet from the lumen. The same may be true for even a clearance fit if a portion of the stylet exterior makes contact with the interior of the tubular body during withdrawal. Accordingly, the change in material type may provide a visual, physical, or audible indication to the user that the distal end of the stylet is in close proximity to the proximal end of the lumen.

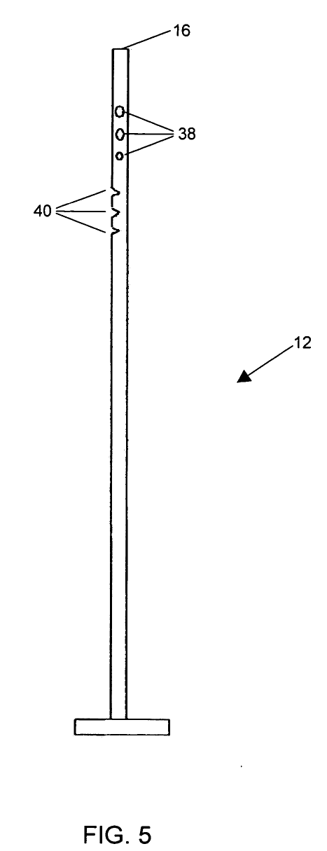

FIG. 5 illustrates two more types of indicators of the present invention. As shown, one type of indicator may be a hole 38 in the stylet 12. The hole 38 may be all the way through the body of the stylet 12. The hole 38 may be visually or physically recognized by the user.

Moreover, the hole 38 may create a change in the exterior of the stylet 12 sufficient to generate a noticeable difference in the level or tone of the sound that is produced from the withdrawal of the stylet 12 from the tubular body. The magnitude of the audibly recognizable noise may increase as the fit between the exterior of the stylet 12 and the interior surface of the tubular body is tightened. For example, an interference fit may result in a larger noise being created than a clearance fit. Moreover, if a number of holes are used for the indicator, the magnitude of the audibly recognizable noise as the user withdraws the stylet 12 from the lumen may be further increased, or the tone altered. Accordingly, a hole 38 may provide a visual, physical, or audible indication to the user that the distal end 16 of the stylet 12 is in close proximity to the proximal end of the lumen.

FIG. 5 also illustrates that another type of indicator may be an indentation 40 into the exterior of the stylet 12. The indentation 40 may be visually or physically recognizable. Moreover, the indentation 40 may create a change in the exterior of the stylet 12 sufficient to generate a noticeable difference in the level or tone of the sound that is produced from the withdrawal of the stylet 12 from the tubular body. As the indentation 40 is removed from the lumen, it may interact with the interior surface of the tubular body in a such a manner to create an audibly recognizable noise. The magnitude of the audibly recognizable noise may increase as the fit between exterior of the stylet 12 and the interior of the lumen is tightened. For example, an interference fit may result in a larger noise being created than a clearance fit. Moreover, if a number of indentations 40 are used for the indicator, the magnitude of the audibly recognizable noise as the user withdraws the stylet 12 from the lumen may be further increased, or the tone altered. Accordingly, an indentation 40 may provide a visual, physical, or audible indication to the user that the distal end 16 of the stylet 12 is in close proximity to the proximal end of the lumen.

Another type of indicator of the present invention may be a sandblasting of at least a portion of the exterior of the stylet. Sandblasting may distort the color of at least a portion of the exterior of the stylet, such as producing a hazy appearance. Sandblasting also may alter the abrasiveness of the exterior of the stylet. Hence, sandblasting at least a portion of the exterior of the stylet may provide a noticeable change in the exterior of the stylet that may be visually or physically recognized by the user.

The change in the abrasiveness of the stylet exterior also may create a noticeable difference in the level or tone of the sound that is produced from the withdrawal of the stylet from the tubular body if a portion of the stylet exterior makes contact with the interior of the tubular body during withdrawal. According, sandblasting at least a portion of the exterior of the stylet may provide a visual, physical, or audible indication to the user that the distal end of the stylet is in close proximity to the proximal end of the lumen.

Another type of indicator of the present invention may be a coating of at least a portion of the exterior of the stylet. Coating a portion of the stylet may produce a difference in color or the abrasiveness of the exterior of the stylet. Hence, coating at least a portion of the exterior of the stylet may provide a noticeable change in the exterior of the stylet that may be visually or physically recognized by the user.

The change in the abrasiveness of the stylet exterior also may create a noticeable difference in the level or tone of the sound that is produced from the withdrawal of the stylet from the tubular body if a portion of the stylet exterior makes contact with the interior of the tubular body during withdrawal. According, coating at least a portion of the exterior of the stylet may provide a visual, physical, or audible indication to the user that the distal end of the stylet is in close proximity to the proximal end of the lumen.

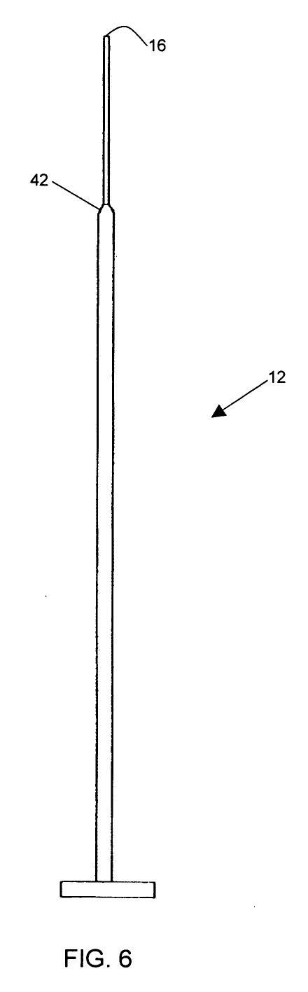

FIG. 6 illustrates another type of indicator of the present invention. As shown, the indicator may be either a reduction 42 or an expansion in the outer diameter of the stylet 16. The reduction 42 or expansion in the circumference of the stylet 16 may provide the user with a noticeable change in the size of the stylet 16 that may be visually or physically recognizable.

Additionally, the reduction or expansion in the outer diameter of the stylet 16 may create a noticeable difference in the level or tone of the sound that is produced from the withdrawal of the stylet from the tubular body if a portion of the stylet exterior makes contact with the interior of the tubular body before the reduction or after the expansion. For example, going from an interference fit between the stylet exterior and the boundary of the lumen to an clearance fit due to a reduction in the outer diameter of the stylet, or vice versa due to an expansion in the outer diameter, may increase or decrease the level of noise created as the stylet is withdrawn, or change its tone. Accordingly, the reduction 42 or expansion in the outer diameter of the stylet may provide a visual, physical, or audible indication to the user that the distal end 16 of the stylet 12 is in close proximity to the proximal end of the lumen.

In another embodiment of the present invention, the stylet may have more than one indicator that the stylet is about to be completely withdrawn from the lumen during use. The indicators may be located in the proximity of the distal end of the stylet. Again, the type of indicators may include one or more of each of the following: (1) ink markings, (2) laser etchings, (3) changes in the material type of the stylet, (4) holes, (5) indentations into the exterior of the stylet, (6) sandblastings of at least a portion of the exterior of the stylet, (7) coatings of at least a portion of the exterior of the stylet, or (8) reductions or expansions in the outer diameter of the stylet.

The indicators selected also may include multiple indicators of the same type as identified directly above. For example, the indicators selected may be a plurality of ink markings, a plurality of laser etchings, a plurality of changes in the type of material of the stylet, a plurality of holes, etc. FIG. 3 illustrates a plurality of ink markings 34. Additionally, FIG. 4 shows a plurality of laser etchings 36.

Alternatively, the indicators selected may include at least two of the eight different types of indicators identified above. For example, the indicators selected may be a combination of an ink marking with a laser etching. Further illustrative combinations include the following combinations: (1) an ink marking and a change in the type of material, (2) a laser etching and a hole, (3) an indentation into the exterior of the style and sandblasting of at least a portion of the exterior of the stylet, and (4) coating at least a portion of the exterior of the stylet and a reduction in the outer diameter of the stylet. As one skilled in the art would understand, other possible combinations may include one of each of the eight types of indicators identified above with any one of the remaining types of indicators discussed.

In another alternative, the indicators selected may include three or more of the eight different types of indicators identified above. For example, the indicators selected may be a combination including an ink marking, a laser etching, and a change in the type of material. As one skilled in the art would understand, other possible combinations may include three or more any of the different eight types of indicators. In other words, the different types of indicators are all interchangeably usable in combination with each other.

In yet another alternative, the indicators selected may again include more than one of the eight different types of indicators identified above. Additionally, one of the different types selected may include a plurality of that type of indicator. For example, the indicators selected may be a combination of two or more ink markings with a laser etching, or vice versa. Further illustrative combinations include the following combinations: (1) two or more ink markings and a change in the type of material, (2) two or more laser etchings and a hole, (3) two or more indentations into the exterior of the style and sandblasting of at least a portion of the exterior of the stylet, or (4) vice versa. As one skilled in the art would understand, other possible combinations may include a plurality of any one of the eight types of indicators identified above with any one of the remaining types of indicators discussed.

In yet another alternative, the indicators selected may include more than one of the eight different types of indicators identified above. Additionally, each of the different types selected may include a plurality of that type of indicator. For example, the indicators selected may be a combination of two or more ink markings with two or more laser etchings, or vice versa. Further illustrative combinations include the following combinations: (1) two or more ink markings and two or more changes in the type of material, (2) two or more laser etchings and two or more holes, and (3) two or more indentations into the exterior of the style and two or more sandblastings of at least a portion of the exterior of the stylet. As one skilled in the art would understand, other possible combinations may include a plurality of any one of the eight types of indicators identified above with a plurality of any one of the remaining types of indicators previously discussed. As an illustrative example, FIG. 5 shows a plurality of holes 38 combined with a number of indentations 40.

In another embodiment of the present invention, a method for indicating the longitudinal position of a stylet relative to an elongated medical device, such as a needle or catheter, is provided. The method includes at least partially withdrawing a stylet having a proximal end and a distal end from a longitudinal lumen of an elongated medical device and recognizing at least one indication provided by an indicator located on the stylet in the approximate proximity of the distal end of the stylet.

The method may further include recognizing at least one visual, physical, or audible indication. Visual, physical, or audible indications may be provided by different types of indicators that include ink markings, laser etchings, changes in the material type of the stylet, holes, indentations into the exterior of the stylet, sandblastings of at least a portion of the exterior of the stylet, coatings of at least a portion of the exterior of the stylet, or reductions or expansions in the outer diameter of the stylet, as previously discussed above.

In one embodiment, the apparatus may be used multiple times and the method may be repeatable. For instance, the same stylet and/or medical device may be used several times. Additionally, the method may be a repeatable process involving the removal of a stylet from a medical device and subsequently inserting the stylet into the medical device a number of times, or vice versa.

In another embodiment, one or more indicators may be optimally positioned in the vicinity of the distal end of the stylet. For instance, an indicator may be positioned between 5 and 50 mm from the tip of the distal end of the stylet. In one aspect, the indicator may be positioned between 10 and 20 mm from the tip of the distal end of the stylet. Other distances may be used.

If more than two indicators are used, the indicators may be spaced at a uniform spacing, such as 5, 10, 15, or 20 mm apart. Alternatively, the multiple indicators may be spaced closer together nearer the tip of the distal end of the stylet. The indicators may become more recognizable (or more numerous) as the distal end becomes closer to being fully withdrawn from the medical device. For example, multiple indicators may be spaced 20 mm, 15 mm, 10 mm, and then 5 mm apart to facilitate indicating that the distal end of the stylet is approaching being fully withdrawn from the medical device. In one aspect, indicators may be spaced at 60 mm, 40 mm, 25 mm, 15 mm, 10 mm, and then 5 mm from the distal end of the stylet. Other spacings may be used.

While the preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein.

It is therefore intended that the foregoing detailed description be regarded as illustrative rather than limiting, and that it be understood that it is the following claims, including all equivalents, that are intended to define the spirit and scope of this invention.

1. A medical system comprising:
an elongated medical device with a flexible tubular body having a longitudinal lumen extending through a portion thereof; and
a stylet configured to be inserted into and subsequently removed from the longitudinal lumen and operable to steer an end of the flexible tubular body to a desired location within a patient, the stylet having a proximal end, a distal end, and at least one indicator located in the approximate proximity of the distal end, wherein as the stylet is being withdrawn from the longitudinal lumen the at least one indicator indicates that the distal end of the stylet is about to exit the longitudinal lumen.

2. The medical system of claim 1, wherein the elongated medical device is a catheter and the at least one indicator is located within approximately 50 mm of the distal end.

3. The medical system of claim 1, wherein the elongated medical device is a needle and the at least one indicator is located within approximately 50 mm of the distal end.

4. The medical system of claim 1, comprising a plurality of indicators and the stylet is configured to have a cylindrical exterior with a uniform circular cross-section.

5. The medical system of claim 1, wherein the at least one indicator comprises an ink marking.

6. The medical system of claim 1, wherein the at least one indicator comprises a laser etching.

7. The medical system of claim 1, wherein the at least one indicator comprises a change in material type of the stylet.

8. The medical system of claim 1, wherein the at least one indicator comprises a hole.

9. The medical system of claim 1, wherein the at least one indicator comprises an indentation into an exterior of the stylet.

10. The medical system of claim 1, wherein the at least one indicator comprises sandblasting at least a portion of an exterior of the stylet.

11. The medical system of claim 1, wherein the at least one indicator comprises coating at least a portion of an exterior of the stylet.

12. The medical system of claim 1, wherein the at least one indicator comprises a reduction or an expansion in the outer diameter of the stylet.

13. A medical device comprising:
a stylet having a proximal end and a distal end, the stylet being configured to be inserted into and longitudinally movable within a longitudinal lumen of an elongated medical apparatus, the elongated medical apparatus having a flexible body such that the stylet is operable to function as guidewire and steer an end of the elongated medical apparatus to a desired location within a patient; and
at least one indicator located on the stylet in the approximate proximity of the distal end, wherein the at least one indicator indicates that the distal end of the stylet is about to exit the longitudinal lumen as the stylet is being withdrawn from the longitudinal lumen.

14. The medical device of claim 13, wherein the at least one indicator is a means for visual notification that the distal end of the stylet is about to exit the longitudinal lumen as the stylet is being withdrawn from the longitudinal lumen.

15. The medical device of claim 14, comprising a plurality of means for visual notification that the distal end of the stylet is about to exit the longitudinal lumen as the stylet is being withdrawn from the longitudinal lumen.

16. The medical device of claim 15, wherein the at least one indicator is a means for physical notification that the distal end of the stylet is about to exit the longitudinal lumen as the stylet is being withdrawn from the longitudinal lumen.

17. The medical device of claim 13, wherein the at least one indicator is a means for audible notification that the distal end of the stylet is about to exit the longitudinal lumen as the stylet is being withdrawn from the longitudinal lumen.

18. A method for indicating the longitudinal position of a stylet relative to an elongated medical device comprising:
at least partially withdrawing a stylet having a proximal end and a distal end from a longitudinal lumen of an elongated medical device with a flexible tubular body, the stylet being a guidewire operable to steer a second distal end of the flexible tubular body to a desired location within a patient; and
recognizing at least one indication provided by an indicator located on the stylet in the approximate proximity of the distal end, wherein the indicator indicates that the distal end of the stylet to is about to exit the elongated medical device if the stylet is continued to be withdrawn from the longitudinal lumen.

19. The method of claim 18, wherein recognizing at least one indication provided by an indicator located on the stylet in the approximate proximity of the distal end comprises visually recognizing at least one indication.

20. The method of claim 18, wherein recognizing at least one indication provided by an indicator located on the stylet in the approximate proximity of the distal end comprises physically recognizing at least one indication.

21. The method of claim 18, wherein recognizing at least one indication provided by an indicator located on the stylet in the approximate proximity of the distal end comprises audibly recognizing at least one indication.
